(19) United States
(12) Patent Application Publication
(10) Pub. No.: US 2026/0105994 A1
(43) Pub. Date: Apr. 16, 2026

(54) METHOD FOR EVALUATING AND PREDICTING COMPREHENSIVE TOXICITY OF FLUE GAS POLLUTANTS BASED ON GRAPH NEURAL NETWORK

(71) Applicant: Tianjin University Of Commerce, Tianjin (CN)

(72) Inventors: Yunan SUN, Tianjin (CN); Guanyi CHEN, Tianjin (CN); Beibei YAN, Tianjin (CN); Mingya ZHU, Tianjin (CN); Junyu TAO, Tianjin (CN); Lan MU, Tianjin (CN); Shuang WU, Tianjin (CN); Zhuo CUI, Tianjin (CN); Xiaohua WANG, Tianjin (CN)

(21) Appl. No.: 19/010,446

(22) Filed: Jan. 6, 2025

(30) Foreign Application Priority Data
Oct. 16, 2024 (CN) 202411458829.6

(51) Int. Cl. G16C 20/30; G16C 20/70

(52) U.S. Cl. CPC G16C 20/30; G16C 20/70

(57) ABSTRACT

A method for evaluating and predicting comprehensive toxicity of flue gas pollutants based on graph neural network is provided, including following steps: determining a chemical composition and concentration data of the flue gas pollutants, and constructing a target flue gas pollution data set; inputting the target flue gas pollution data set into a compound property prediction model, and predicting and outputting prediction features of a target compound; inputting the prediction features of the target compound into an interaction relationship prediction model, outputting interaction information between a compound and the biological targets, and storing the interaction information in a structured way to generate a target response matrix; and calculating a target flue gas comprehensive response score based on the target response matrix, standardizing a response value of each target, and combining a weight of each target to obtain a final score, generating an interaction relationship network.

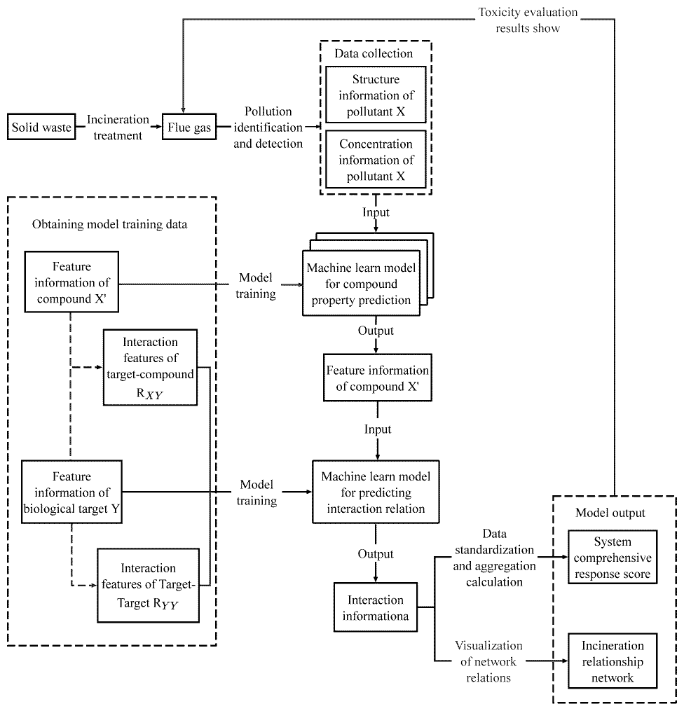

### CROSS-REFERENCE TO RELATED APPLICATIONS

This application claims priority to Chinese Patent Application No. 202411458829.6, filed on Oct. 16, 2024, the contents of which are hereby incorporated by reference.

### TECHNICAL FIELD

The disclosure relates to the technical field of toxicity evaluation and prediction of environmental pollutants, and in particular to a method for evaluating and predicting comprehensive toxicity of flue gas pollutants based on graph neural network.

### BACKGROUND

Solid waste incineration treatment technology, as a widely used waste treatment method, has the advantages of significantly reducing waste volume and reusing resources through heat energy recovery. However, there are many kinds of pollutants produced during incineration, such as dioxins, heavy metals, polycyclic aromatic hydrocarbons (PAHs), etc., which are complex in nature and pose a potential threat to the environment and human health. If these pollutants are not properly assessed and managed, the pollutants may lead to serious environmental pollution and health risks. Accurate evaluation of the toxicity of incineration pollutants is therefore essential for environmental protection and public health.

At present, the research on the mechanism of environmental toxicity of compounds is mainly focused on a single compound or a simple compound group, and the toxicity evaluation method for complex mixtures is not mature, which shows obvious limitations in dealing with complex mixtures produced by incineration. Existing evaluation methods primarily rely on empirical conclusions drawn from a large number of experimental data. Predicting based on apparent toxicity is usually time-consuming and costly, and lacks in-depth research on molecular interaction. For example, traditional prediction methods such as quantitative structure-activity relationship (QSAR) have limited applicability to complex mixtures. This limitation compromises the reliability and accuracy of the predictions and makes it difficult to reveal the molecular mechanism of toxicity.

Under realistic environmental conditions, pollutants produced by incineration often exist in the form of complex mixtures. The components in these mixtures may interact with each other, resulting in the overall toxicity different from the simple addition of the toxicity of each component. These interactions may lead to synergistic or antagonistic effects, and it is difficult to accurately predict the comprehensive toxicity of mixtures by traditional toxicity evaluation methods of single compounds. Therefore, it is particularly important to establish a scientific method to evaluate the overall toxicity of the mixture.

In order to truly understand and predict the toxicity of complex mixtures, it is necessary to deeply study the interaction mechanism at the molecular level. By analyzing the biochemical basis of interactions between compounds, a deeper understanding of toxicity is provided. Based on this, the complex interaction between molecular structure and biological targets are captured through advanced machine learning methods such as graph neural network, and then develop more accurate prediction models and evaluation methods, which provides an innovative path for comprehensive toxicity evaluation of complex mixtures, particularly in the context of incineration pollutants.

### SUMMARY

In order to solve the problems in the prior art, the disclosure provides a method for evaluating and predicting comprehensive toxicity of flue gas pollutants based on graph neural network, and deeply analyzes the toxicity of mixed pollutants through molecular level analysis.

In order to achieve the above purpose, the disclosure provides a method for evaluating and predicting comprehensive toxicity of flue gas pollutants based on graph neural network, including the following steps:
determining a chemical composition and concentration data of the flue gas pollutants, and constructing a target flue gas pollution data set;
inputting the target flue gas pollution data set into a compound property prediction model, and predicting and outputting prediction features of a target compound;
inputting the prediction features of the target compound into an interaction relationship prediction model, combining feature information of biological targets to output interaction information between a compound and the biological targets, and storing the interaction information in a structured way to generate a target response matrix;
calculating a target flue gas comprehensive response score based on the target response matrix, standardizing a response value of each target, and combining a weight of each target to obtain a final score, generating an interaction relationship network, where the interaction relationship network displays an interaction between the compound and the biological targets and an interaction between targets through visualization;
where, the flue gas pollutants are flue gas pollutants generated by solid waste incineration treatment; the compound property prediction model is constructed by a machine learning algorithm, and is obtained by training with molecular structure information and known features in a similar compound set; compound features and biological target features are input as node features, input by compound features and biological target features as node features, and compound-target features and target-target features form edge features of the model to be input.

Optionally, constructing the target flue gas pollution data set includes:
determining a chemical composition and content of pollutants by an on-line detection technology, a flue gas sampling analysis and a computer simulation method;
where a chemical composition information set of the flue gas pollutants is S={s₁, s₂, . . . , sₙ}, where each element sᵢ represents chemical structure information of a specific pollutant, including a name, a chemical structure representation, a chemical formula and a molecular weight of the compound;
a concentration information constitution set of the flue gas pollutants is C={c₁, c₂, . . . , cₙ}, and each element cᵢ corresponds to a concentration of sᵢ in an effluent in a chemical composition set S, and a time and a place of sample collection are recorded to obtain the target flue gas pollution data set.

Optionally, obtaining the compound property prediction model includes:
constructing a training data set;
constructing an initial compound property prediction model by using the machine learning algorithm, taking known feature data in the similar compound set in the training data set as the target output, training the initial compound property prediction model to learn a mapping relationship from structure to property, optimizing a model through a feature extraction step, optimizing model parameters and evaluating performance, and obtaining an optimized compound property prediction model by adopting cross-validation and independent test set validation methods;
where in a process of model training, Bayesian optimization or a grid search method is combined to adjust superparameters.

Optionally, constructing the training data set includes:
through methods of chemical database retrieval, literature review, computer-aided chemical synthesis and simulation analysis, constructing a similar compound feature set X′ with a similar structure to the chemical composition information set S, and defining a similar compound feature set X′={x′₁, x′₂, . . . , x′ₙ}, where each element x′; represents features of a screened compound, including chemical structure information, chemical bond features, atomic features, functional group features and physical and chemical properties thereof;
obtaining basic information of a protein and interaction data between the compound and the targets by database retrieval and the literature review, and supplementing binding loci and binding affinity between the targets and the compound by combining a computer-aided chemical simulation and experimental results, and defining a target feature set Y={y₁, y₂, . . . , yₖ} to obtain biological target data, where the target feature set includes protein target features possibly influenced by the target compound, including a protein sequence, a three-dimensional structure, functional domain information and interaction information with the compound;
extracting the interaction information between the compound and the biological targets, and constructing a first interaction set R_{xy}={(x′ᵢ, yⱼ, aᵢⱼ)|x′ᵢ∈X′, yⱼ∈Y}, where an element aᵢⱼ describes interaction between the compound x′ᵢ and the targets yⱼ, and a set R_{XY} includes binding affinity, binding loci description and reaction path; and
extracting protein-protein interaction information between target feature sets Y, and constructing a second interaction set R_{YY}={(yⱼ, yₖ, iⱼₖ)|yⱼ, yₖ∈Y}, where each element iⱼₖ describes interaction between targets yⱼ and yₖ.

Optionally, in a process of constructing the training data set, if data missing or abnormal values occur, a data completion algorithm is adopted for completion, and extreme values are identified and processed by an abnormal value detection mechanism; in order to cope with a situation of rare or extreme pollutants, the evaluation ability of uncommon pollutants is improved by adding an abnormal detection module and a retraining mechanism.

Optionally, the interaction relationship prediction model adopts the graph neural network, and compound node features and the biological target features are input as the node features, and compound-target features and target-target features form the edge features to be input for training and optimization, and after the training and optimization, the model outputs the interaction information of biological target system under a mixture action;
where, the compound node features are formed by the prediction features of the target compound;
the biological target features are extracted from the target feature set, including structure information, functional domain features and known biological activity data of the biological target;
the compound-target features come from the first interaction set R_{XY}, including the binding affinity, binding loci and the reaction path between each compound and corresponding targets; and
the target-target features use the protein-protein interaction information in the second interaction set R_{YY} to describe an interaction relationship and signal path between the targets.

Optionally, the interaction information includes:
changes of target activity: used for quantitative evaluation of target function influence;
signal path disturbance: used to reveal whether a biological signal path is affected by a mixture;
network reconfiguration information: used to show the changes of interaction between the targets and reflect influence of the mixture on network structure reconfiguration.

Optionally, a method for calculating the target flue gas comprehensive response score includes:

$${S}_{system} = {\sum }_{i = 1}^{n} {w}_{i} \cdot {\sum }_{j = 1}^{m} {r}_{ij}^{′}$$

in the formula, r′ᵢⱼ is a response value of the standardized i-th target yᵢ under a response variable j, wᵢ is a weight of target i, S_{system} is the target flue gas comprehensive response score, n represents a total number of the targets in a system, and m represents a total number of response variables of each target.

Optionally, the interaction relationship network is represented by nodes and edges, and displayed by visual tools;
where, the nodes represent the compounds or the targets, including reactivity, biological activity and concentration feature information of the compounds, as well as the structure information and the functional domain features of the targets; and
the edges indicate the interaction between the compounds and the targets, or the interaction between the targets and the targets, and edge information includes the binding affinity, the binding loci and the interaction path.

Compared with the prior art, the disclosure has the following advantages and technical effects.

First, through the combined design of two layers of machine learning, the disclosure realizes the prediction of the comprehensive influence of complex molecular structural properties and mixtures on the target network. Compared with the existing toxicity evaluation method of a single compound, the method of the disclosure may not only comprehensively consider the target features and interaction, but also significantly improve the prediction accuracy of the comprehensive toxicity of a complex mixture, may more accurately capture the synergistic and antagonistic effects in the mixture, and may also directly realize the rapid prediction from the molecular structure to the biological effects.

Second, the disclosure reduces the dimension of the high-dimensional response data of the target network, simplifies it into a comprehensive score, and provides an intuitive and quantitative pollution intensity evaluation method. Through data visualization tools, the complex compound-target network interaction relationship is intuitively displayed in the form of network diagram. This evaluation system provides new ideas and methods for the toxicity evaluation of environmental pollutant mixtures, enhances researchers' ability to understand and analyze data, and improves the efficiency of decision support.

Third, the method is used for calculating and predicting the toxicity of the mixture by combining the data format of edge and node features, so that the processing and display of complex data are more convenient and efficient.

Fourth, the disclosure provides a more comprehensive and in-depth analysis for the toxicity prediction of the mixture, which may not only accurately evaluate the toxicity of the mixture under laboratory conditions, but also play an important role in actual environmental monitoring and pollution control.

### BRIEF DESCRIPTION OF THE DRAWINGS

The accompanying drawings, which constitute a part of this application, are used to provide a further understanding of this application. The illustrative embodiments of this application and their descriptions are used to explain this application, and do not constitute an improper limitation of this application.

The FIGURE is a flowchart of a method for evaluating and predicting comprehensive toxicity of flue gas pollutants based on graph neural network according to an embodiment of the present disclosure.

### DETAILED DESCRIPTION OF THE EMBODIMENTS

It should be noted that the embodiments in this application and the features in the embodiments may be combined with each other without conflict. The present application will be described in detail with reference to the attached drawings and embodiments.

It should be noted that the steps shown in the flowchart of the accompanying drawings may be executed in a computer system such as a set of computer-executable instructions, and although the logical order is shown in the flowchart, in some cases, the steps shown or described may be executed in a different order from here.

The disclosure provides a method for evaluating and predicting comprehensive toxicity of flue gas pollutants based on graph neural network, as shown in the FIGURE, including:
determining a chemical composition and concentration data of the flue gas pollutants, and constructing a target flue gas pollution data set;
inputting the target flue gas pollution data set into a compound property prediction model, and predicting and outputting prediction features of a target compound;
inputting the prediction features of the target compound into an interaction relationship prediction model, combining feature information of biological targets to output interaction information between a compound and the biological targets, and storing the interaction information in a structured way to generate a target response matrix;
calculating a target flue gas comprehensive response score based on the target response matrix, standardizing a response value of each target, and combining a weight of each target to obtain a final score, generating an interaction relationship network, where the interaction relationship network displays an interaction between the compound and the biological targets and an interaction between targets through visualization;
where, the flue gas pollutants are flue gas pollutants generated by solid waste incineration treatment; the compound property prediction model is constructed by a machine learning algorithm, and is obtained by training with molecular structure information and known features in a similar compound set; compound features and biological target features are input as node features, input by compound features and biological target features as node features, and compound-target features and target-target features form edge features of the model to be input.

Specifically, this example deeply analyzes the toxicity of mixed pollutants from the molecular level. Firstly, the chemical composition and concentration of pollutants discharged from solid waste incineration are determined by detection technology. At the same time, the basic feature information of compounds with similar structures to the detected substances is collected as the database needed to build the model. Based on the relationship between molecular structure information and activity, the features of each single substance in the detected mixture are predicted, and the prediction results and the features of the biological end in the collected data are used to construct an interaction relationship prediction model to predict the comprehensive influence of the mixture on the target network. Through the dimensionality reduction and data visualization of the model output data, the effect of the mixture on the target network is intuitively displayed, and the pollution intensity is quantitatively evaluated by the comprehensive response score.

This multi-level model based on graph neural network may not only systematically evaluate the overall toxicity of mixed pollutants, but also reveal its specific molecular interaction mechanism, providing a more scientific and accurate toxicity evaluation method for environmental protection and public health.

This embodiment is suitable for solid waste incineration plants, environmental monitoring stations and pollution control systems, and may be used to evaluate the comprehensive toxicity of various flue gas pollutants generated in the incineration process to biological systems in real time.

Further, constructing the target flue gas pollution data set includes:
determining a chemical composition and content of pollutants by an on-line detection technology, a flue gas sampling analysis and a computer simulation method;
where a chemical composition information set of the flue gas pollutants is S={s₁, s₂> . . . , sₙ}, where each element sᵢ represents chemical structure information of a specific pollutant, including a name, a chemical structure representation, a chemical formula and a molecular weight of the compound;
a concentration information constitution set of the flue gas pollutants is C={c₁, c₂, . . . , cₙ}, and each element cᵢ corresponds to a concentration of sᵢ in an effluent in a chemical composition set S, and a time and a place of sample collection are recorded to obtain the target flue gas pollution data set.

Specifically, in this embodiment, pollutants X are defined as flue gas pollutants generated by solid waste incineration treatment, and the chemical composition and content of pollutant X are determined by on-line detection technology, the flue gas sampling analysis and the computer simulation method and other technologies.

A chemical composition information set of the flue gas pollutants is S={s₁, s₂, . . . , sₙ}, in which each element sᵢ represents the chemical structure information of a specific pollutant, including the name, chemical structure representation (such as SMILES or InChI format), chemical formula and molecular weight of the compound.

The concentration information of flue gas pollutants forms a set C={c₁, c₂, . . . , cₙ}, and each element cᵢ corresponds to the concentration (unit: microgram per cubic meter, μg/m3) in the effluent in the chemical composition set S, and the time and place of sample collection are recorded as the compound structure information and concentration data set input by the system model.

Further, obtaining the compound property prediction model includes:
constructing a training data set;
constructing an initial compound property prediction model by using the machine learning algorithm, taking known feature data in the similar compound set in the training data set as the target output, training the initial compound property prediction model to learn a mapping relationship from structure to property, optimizing a model through a feature extraction step, optimizing model parameters and evaluating performance, and obtaining an optimized compound property prediction model by adopting cross-validation and independent test set validation methods;
where in a process of model training, Bayesian optimization or a grid search method is combined to adjust superparameters.

Specifically, based on the corresponding relationship between molecular structure information and activity, the compound property prediction model is constructed. This model uses the molecular structure information and known features in similar compound set X, and trains the model through machine learning algorithm to predict the compound feature X of target compound set S. For each compound x′ᵢ in the set X′, the chemical structure information and known physical and chemical features are used to construct the input feature matrix of the model.

In the model training stage, the known feature data in the similar compound set X′ is used as the target output, and the training model learns the mapping relationship from structure to property. The training process of model includes feature extraction, model optimization and performance evaluation. Cross validation and test set validation are used to evaluate the prediction accuracy and generalization ability of the model. The model is trained and verified to be effective, that is, the model may be used to predict the features of the target compound set. By inputting the compound structure information in the set S, the model may output the feature prediction X={x₁, x₂, . . . , xₙ} of the target compound set S. These predictive features include important properties such as reactivity, stability and biological activity of compounds, which are used as input data for subsequent mixed toxicity evaluation.

Further, constructing the training data set includes:
through methods of chemical database retrieval, literature review, computer-aided chemical synthesis and simulation analysis, constructing a similar compound feature set X′ with a similar structure to the chemical composition information set S, and defining a similar compound feature set X′={x′₁, x′₂, . . . , x′ₙ}, where each element x′ᵢ represents features of a screened compound, including chemical structure information, chemical bond features, atomic features, functional group features and physical and chemical properties thereof;
obtaining basic information of a protein and interaction data between the compound and the targets by database retrieval and the literature review, and supplementing binding loci and binding affinity between the targets and the compound by combining a computer-aided chemical simulation and experimental results, and defining a target feature set Y={y₁, y₂, . . . , yₖ} to obtain biological target data, where the target feature set includes protein target features possibly influenced by the target compound, including a protein sequence, a three-dimensional structure, functional domain information and interaction information with the compound;
extracting the interaction information between the compound and the biological targets, and constructing a first interaction set R_{xy}={x′ᵢ, yⱼ, aᵢⱼ)|x′ᵢ∈X′, yⱼ∈Y}, where an element aᵢⱼ describes interaction between the compound x′ᵢ and the targets yⱼ, and a set R_{xy} includes binding affinity, binding loci description and reaction path; and
extracting protein-protein interaction information between target feature sets Y, and constructing a second interaction set R_{YY}={(yⱼ, yₖ, iⱼₖ)|yⱼ, yₖ∈Y}, where each element iⱼₖ describes interaction between targets yⱼ and yₖ.

Specifically, after obtaining the target flue gas pollution data set, the data required by the compound property prediction model, that is, the training data set, is obtained by constructing a similar compound set X′.

By searching chemical databases (such as PubChem, ChEMBL), literature review, computer-aided chemical synthesis and simulation analysis, a set X′ of compound features with similar structure to the target compound set S is constructed (Tanimoto coefficient and other similarity indicators are used to ensure that the selected compound had high similarity with the target compound in structure)

Similar compound feature set X′={x′₁, x′₂, . . . , x′ₙ} is defined, where each element x′ᵢ represents the features of the screened compound, including detailed chemical structure information, chemical bond features, atomic features, functional group features and the physical and chemical properties (such as molecular weight, polarity, solubility, melting point, etc.). These data are standardized and formatted to support the training of subsequent models.

At the same time, the target feature set Y={y₁, y₂, . . . , yₖ} is defined, and the biological target data are obtained through protein databases (such as UniProt, PDB), biological experimental determination, literature research and other channels. This set contains protein target features that may be affected by the target compound, including protein sequence, three-dimensional structure, functional domain information and interaction information with the compound. These data are the basis for understanding the possible influence of the compound in the biological system.

Further extracting the interaction information between the compound and the biological target, and defining a first interaction set R_{xy}={x′, yⱼ, aᵢⱼ)|x′ᵢ∈X′, yⱼ∈Y}, where the element aᵢⱼ description describes the interaction between the compound x′ᵢ and the target yⱼ. This information includes binding affinity (such as IC50, EC50), description of binding loci and reaction paths. In addition, the protein-protein interaction information between target Y is extracted, and a second interaction set R_{YY}={(yⱼ, yₖ, iⱼₖ)|yⱼ, yₖ∈Y} is constructed, in which each element iⱼₖ describes the interaction between target yⱼ and target yₖ, and the interaction information is helpful to understand how compounds regulate biological processes and possible biological effects by influencing protein network.

In the data collection stage, various systematic methods of data collection and interactive information extraction are used to provide the data basis for the subsequent compound feature prediction model and model training.

Furthermore, in a process of constructing the training data set, if data missing or abnormal values occur, a data completion algorithm is adopted for completion, and extreme values are identified and processed by an abnormal value detection mechanism; in order to cope with a situation of rare or extreme pollutants, the evaluation ability of uncommon pollutants is improved by adding an abnormal detection module and a retraining mechanism.

Further, the interaction relationship prediction model adopts the graph neural network, and compound node features and the biological target features are input as the node features, and compound-target features and target-target features form the edge features to be input for training and optimization, and after the training and optimization, the model outputs the interaction information of biological target system under a mixture action;
where, the compound node features are formed by the prediction features of the target compound;
the biological target features are extracted from the target feature set, including structure information, functional domain features and known biological activity data of the biological target;
the compound-target features come from the first interaction set R_{XY}, including the binding affinity, binding loci and the reaction path between each compound and corresponding targets; and
the target-target features use the protein-protein interaction information in the second interaction set R_{YY} to describe an interaction relationship and signal path between the targets.

Specifically, in the model design, the model inputs the features of compounds and biological targets as node features, and the features of compounds-targets and features of targets-targets constitute the edge features of the model for inputting and processing.

In this embodiment, the compound node features are composed of the feature set X output by the compound property prediction model. At the same time, these features are combined with the concentration information of compounds in the environment C, which provides a complete description for the state of plant compounds incinerated by solid waste in a specific environment.

The features of biological targets include structure information, functional domain features and known biological activity data of biological targets, which are extracted from the set Y of biological targets, providing a basis for understanding the mechanism of action of compounds on specific targets.

The compound-target features come from the first interaction set R_{XY}, which contains the binding affinity, binding loci and reaction path between each compound and its corresponding target. These edge features reflect the direct interaction between compounds and biological targets.

Target-target features describe the interaction relationship and signal pathway between targets by using the protein-protein interaction information in the second interaction set R_{YY}.

By constructing such a complex heterograph structure, the overall interaction between compounds and biological target network is comprehensively analyzed, the local and global features of the network are learned, and the comprehensive influence of compounds on the target network is captured.

Further, the interaction information includes:
changes of target activity: used for quantitative evaluation of target function influence;
signal path disturbance: used to reveal whether a biological signal path is affected by a mixture;
network reconfiguration information: used to show the changes of interaction between the targets and reflect influence of the mixture on network structure reconfiguration.

Specifically, by constructing a complex heterogeneous graph structure, the overall interaction between compounds and biological target network is comprehensively analyzed, the local and global features of the network are learned, and the comprehensive influence of compounds on the target network is captured. After training and optimization, the model outputs the whole interaction information of biological target system under the mixture action, which mainly includes the changes of target activity, signal path disturbance and network reconfiguration information. The changes of target activity provide a quantitative evaluation of the target function influence, while the signal path disturbance reveals which biological signal pathways are significantly affected by the mixture. Network reconfiguration information shows the change of interaction between targets and reflects the influence of mixture on network structure reconfiguration.

Further, the data in the interaction information is stored in a structured way, and the target response matrix is output, specifically:

In order to ensure the comprehensiveness and visualization of the output data, the data output design of the model includes detailed node information and edge information. These data are stored in a structured format (such as JSON or CSV) for further processing and analysis. It mainly includes the following information:

### (1) Node Information:

1) Compound nodes: each compound node contains the following detailed information:
Node ID: unique identifier used to distinguish different compounds.
Name: name of the compound.
Structure: chemical structure representation.
Reactivity: the reactivity index of a compound.
Stability: the stability index of a compound.
Biological activity: biological activity data of compounds.
Concentration information: the concentration of the compound in the environment, in μg/m3.
2) Target nodes: each biological target node contains the following detailed information:
Node ID: a unique identifier used to distinguish different targets.
Name: target name.
Structure information: protein sequence or structure information.
Functional domain features: name and location of functional domain.
Known biological activity: biological activity data of target.

### (2) Side Information:

1) Compound-target edge: each edge describes the interaction features between the compound and the target, including:
Starting node ID: the unique identifier of the compound node.
End node ID: the unique identifier of the target node.
Binding affinity: such as IC50 and EC50.
Binding loci: a detailed description of the binding loci.
Reaction path: a detailed description of the interaction path.
2) Target-target edge: each edge describes the interaction features between targets, including:
Starting node ID: the unique identifier of the first target node.
End node ID: the unique identifier of the second target node.
Interaction strength: the interaction strength between targets.
Signal path information: the name of the signal path involved and its influence.

Furthermore, the target response matrix is used to store the response results of the target under different response conditions, which may take a variety of data representation forms, such as matrix, vector or other structured data structures. The specific format is not limited to one, but a suitable structure is adopted to express the multi-dimensional attributes of the data according to actual needs.

In this embodiment, the target response matrix is:

$$R = [ \begin{matrix}{r}_{11} & {r}_{12} & … & {r}_{1 m} \\ {r}_{21} & {r}_{22} & … & {r}_{2 m} \\ ⋮ & ⋮ & ⋱ & ⋮ \\ {r}_{n 1} & {r}_{n 2} & … & {r}_{nm}\end{matrix} ]$$

In the formula, the element rᵢⱼ represents the response result of the target yᵢ under a specific response variable j;
element rᵢⱼ is a vector or data structure containing multidimensional information, and the attributes of element rᵢⱼ include response value, response type, numerical range, unit, time point and experimental conditions.

Specifically, the target response matrix is used to quantitatively describe the overall response of biological targets under the influence of mixtures. The target response matrix R consists of n×m elements rᵢⱼ, in which: row (n) represents different target y₁, y₂, . . . , yₙ, such as specific protein, receptor, enzyme, etc. Column (m) represents different response variables, such as target activity change, signal path disturbance, gene expression change, etc.

The response value is the prediction result of machine learning model, which is used to quantitatively describe the activity increase or decrease or signal disturbance degree of target yᵢ under the response variable j.

The response type further describes the biological properties of the response, such as activation, inhibition or binding, so as to accurately reflect the biological mechanism in the comprehensive score calculation;
the numerical range defines the possible range of response values to ensure consistency when comparing different response variables;
the unit information provides a dimensional reference for the response value, such as probability or signal strength, to ensure the accuracy in data processing;
the time point attribute is used to indicate the time node where the response occurs, and supports the analysis of dynamic changes;
experimental conditions record the environment or experimental conditions when the response occurs, such as temperature and pH value, which provides an important reference for data interpretation and model adjustment.

Further, the method for calculating the target flue gas comprehensive response score is as follows:
the response variables (such as activity change and signal path disturbance) of each target in the output target response matrix are standardized and weighted and accumulated to obtain the final score:

$${S}_{system} = {\sum }_{i = 1}^{n} {w}_{i} \cdot {\sum }_{j = 1}^{m} {r}_{ij}^{′}$$

in the formula, r′ᵢⱼ is a response value of the standardized i-th target y; under a response variable j, wᵢ is a weight of target i, S_{system} is the target flue gas comprehensive response score, n represents a total number of the targets in a system, and m represents a total number of response variables of each target.

Specifically, the target flue gas comprehensive response score is a quantitative evaluation of the overall response of target network under the action of compound mixture. The score is calculated by integrating the variables such as activity change and signal path disturbance in the target response matrix. The output aims to provide a numerical index to reflect the potential impact of the mixture on the biological system.

The target flue gas comprehensive response score may be a floating point number or an integer, depending on the required accuracy. If a unified range is needed, the score is limited to the range of [0,100] in this embodiment, and the score may intuitively convey the toxicity of the mixture, and a higher value means that the system is greatly affected and the potential toxicity is high; on the contrary, a lower value means that the system is less affected and less toxic.

Furthermore, the interaction relationship network is represented by nodes and edges, and displayed by visual tools;
where, the nodes represent the compounds or the targets, including reactivity, biological activity and concentration feature information of the compounds, as well as the structure information and the functional domain features of the targets; and
the edges indicate the interaction between the compounds and the targets, or the interaction between the targets and the targets, and edge information includes the binding affinity, the binding loci and the interaction path.

Specifically, the interaction relationship network shows the interaction between compounds and biological targets and the interaction between targets through visualization. The network is represented by nodes and edges, and the nodes are represented by the structured format file output by the graph neural network model, which contains feature information such as reactivity, biological activity and concentration of the compound, as well as structure information and functional domain features of the target. Edge indicates the interaction between compound and target, or the interaction between targets and targets. Side information includes binding affinity, binding loci, interaction path, etc. The display of visual tools (such as NetworkX, Cytoscape) helps to describe in detail the specific action path and influence mode of each compound in the biological system, and further explains the biological mechanism behind the system score.

The combination of the interaction relationship network and the comprehensive response score of the system not only provides a concise overall toxicity evaluation, but also shows the specific biological interactions in detail, providing a comprehensive analysis tool from macro to micro.

It should be noted that the data representation forms involved in the present disclosure are not limited to specific mathematical structures, such as formulas or matrix forms. In different implementations, related data may be expressed in any suitable form, such as vectors, tables, graphs or other structured or unstructured ways. The formula or matrix is only a representation in the embodiment of the present disclosure, which is intended to facilitate explanation and implementation. What the present disclosure protects is not limited to such representations, but covers all equivalent ways that may achieve the same function. The core of the disclosure lies in the process and method of comprehensive toxicity evaluation of pollutants, rather than a specific data structure or representation method.

The technical scheme of the disclosure focuses on the toxicity evaluation process based on graph neural network, including data collection, model training, toxicity prediction and other steps. The data representation of all these steps may be displayed in an appropriate way according to the needs of practical applications.

The protection scope of the present disclosure is not limited to any specific data structure, and any representation for realizing these technical steps is within the protection scope. The interaction information between compounds and targets involved in the disclosure may be expressed by different data structures, such as database tables, JSON format files, two-dimensional or multi-dimensional matrices, etc. The selection of these data structures does not affect the toxicity evaluation process and method of the present disclosure. Different embodiments may be adjusted and selected according to actual needs, and these adjustments are within the protection scope of the disclosure.

The above is only the preferred embodiment of this application, but the protection scope of this application is not limited to this. Any change or replacement that may be easily thought of by a person familiar with this technical field within the technical scope disclosed in this application should be included in the protection scope of this application. Therefore, the protection scope of this application should be based on the protection scope of the claims.

What is claimed is:

1. A method for evaluating and predicting comprehensive toxicity of flue gas pollutants based on graph neural network, comprising:
determining a chemical composition and concentration data of the flue gas pollutants, and constructing a target flue gas pollution data set;
inputting the target flue gas pollution data set into a compound property prediction model, and predicting and outputting prediction features of a target compound;
inputting the prediction features of the target compound into an interaction relationship prediction model, combining feature information of biological targets to output interaction information between a compound and the biological targets, and storing the interaction information in a structured way to generate a target response matrix;
calculating a target flue gas comprehensive response score based on the target response matrix, standardizing a response value of each target, and combining a weight of each target to obtain a final score, generating an interaction relationship network, wherein the interaction relationship network displays an interaction between the compound and the biological targets and an interaction between targets through visualization;
wherein, the flue gas pollutants are flue gas pollutants generated by solid waste incineration treatment; the compound property prediction model is constructed by a machine learning algorithm, and is obtained by training with molecular structure information and known features in a similar compound set; the interaction relationship prediction model is the graph neural network, compound features and biological target features are input as node features, and compound-target features and target-target features form edge features of the model to be input.

2. The method for evaluating and predicting the comprehensive toxicity of the flue gas pollutants based on the graph neural network according to claim 1, wherein constructing the target flue gas pollution data set comprises:
determining a chemical composition and content of pollutants by an on-line detection technology, a flue gas sampling analysis and a computer simulation method;
wherein a chemical composition information set of the flue gas pollutants is S={s₁, s₂, . . . , sₙ}, wherein each element sᵢ represents chemical structure information of a specific pollutant, comprising a name, a chemical structure representation, a chemical formula and a molecular weight of the compound;
a concentration information constitution set of the flue gas pollutants is C={c₁, c₂, . . . , cₙ}, and each element cᵢ corresponds to a concentration of sᵢ in an effluent in a chemical composition set S, and a time and a place of sample collection are recorded to obtain the target flue gas pollution data set.

3. The method for evaluating and predicting the comprehensive toxicity of the flue gas pollutants based on the graph neural network according to claim 1, wherein obtaining the compound property prediction model comprises:
constructing a training data set;
constructing an initial compound property prediction model by using the machine learning algorithm, taking known feature data in the similar compound set in the training data set as the target output, training the initial compound property prediction model to learn a mapping relationship from structure to property, optimizing a model through a feature extraction step, optimizing model parameters and evaluating performance, and obtaining an optimized compound property prediction model by adopting cross-validation and independent test set validation methods;
wherein in a process of model training, Bayesian optimization or a grid search method is combined to adjust superparameters.

4. The method for evaluating and predicting the comprehensive toxicity of the flue gas pollutants based on the graph neural network according to claim 3, wherein constructing the training data set comprises:
through methods of chemical database retrieval, literature review, computer-aided chemical synthesis and simulation analysis, constructing a similar compound feature set X′ with a similar structure to the chemical composition information set S, and defining a similar compound feature set X′={x′₁, x′₂, . . . , x′ₙ}, wherein each element x′ᵢ represents features of a screened compound, comprising chemical structure information, chemical bond features, atomic features, functional group features and physical and chemical properties thereof;
obtaining basic information of a protein and interaction data between the compound and the targets by database retrieval and the literature review, and supplementing binding loci and binding affinity between the targets and the compound by combining a computer-aided chemical simulation and experimental results, and defining a target feature set Y={y₁, y₂, . . . , yₖ} to obtain biological target data, wherein the target feature set comprises protein target features possibly influenced by the target compound, comprising a protein sequence, a three-dimensional structure, functional domain information and interaction information with the compound;
extracting the interaction information between the compound and the biological targets, and constructing a first interaction set R_{xy}={(x′ᵢ, yⱼ, aᵢⱼ)|x′ᵢ∈X′, yⱼ∈Y}, wherein an element aᵢⱼ describes interaction between the compound x′ᵢ and the targets yⱼ, and a set R_{XY} comprises binding affinity, binding loci description and reaction path; and
extracting protein-protein interaction information between target feature sets Y, and constructing a second interaction set R_{YY}={(yⱼ, yₖ, iⱼₖ)|yⱼ, yₖ∈Y}, wherein each element iⱼₖ describes interaction between the targets yⱼ and yₖ.

5. The method for evaluating and predicting the comprehensive toxicity of the flue gas pollutants based on the graph neural network according to claim 4, wherein in a process of constructing the training data set, if data missing or abnormal values occur, a data completion algorithm is adopted for completion, and extreme values are identified and processed by an abnormal value detection mechanism; in order to cope with a situation of rare or extreme pollutants, an evaluation ability of uncommon pollutants is improved by adding an abnormal detection module and a retraining mechanism.

6. The method for evaluating and predicting the comprehensive toxicity of the flue gas pollutants based on the graph neural network according to claim 4, wherein the interaction relationship prediction model adopts the graph neural network, and compound node features and the biological target features are input as the node features, and the compound-target features and target-target features form the edge features to be input for training and optimization, and after the training and optimization, the model outputs the interaction information of a biological target system under a mixture action;
wherein, the compound node features are formed by the prediction features of the target compound;
the biological target features are extracted from the target feature set, comprising structure information, functional domain features and known biological activity data of the biological target;
the compound-target features come from the first interaction set R_{XY}, comprising the binding affinity, binding loci and the reaction path between each compound and corresponding targets; and
the target-target features use the protein-protein interaction information in the second interaction set R_{YY} to describe an interaction relationship and a signal path between the targets.

7. The method for evaluating and predicting the comprehensive toxicity of the flue gas pollutants based on the graph neural network according to claim 6, wherein the interaction information comprises:
changes of target activity: used for quantitative evaluation of target function influence;
signal path disturbance: used to reveal whether a biological signal path is affected by a mixture;
network reconfiguration information: used to show changes of interaction between the targets and reflect influence of the mixture on network structure reconfiguration.

8. The method for evaluating and predicting the comprehensive toxicity of the flue gas pollutants based on the graph neural network according to claim 1, wherein a method for calculating the target flue gas comprehensive response score comprises:
$${S}_{system} = {\sum }_{i = 1}^{n} {w}_{i} \cdot {\sum }_{j = 1}^{m} {r}_{ij}^{′}$$
in a formula, r′ᵢⱼ is a response value of the standardized i-th target yᵢ under a response variable j, wᵢ is a weight of target i, S_{system} is the target flue gas comprehensive response score, n represents a total number of the targets in a system, and m represents a total number of response variables of each target.

9. The method for evaluating and predicting the comprehensive toxicity of the flue gas pollutants based on the graph neural network according to claim 1, wherein the interaction relationship network is represented by nodes and edges, and displayed by visual tools;
wherein, the nodes represent the compounds or the targets, comprising reactivity, biological activity and concentration feature information of the compounds, as well as the structure information and the functional domain features of the targets; and
the edges indicate the interaction between the compounds and the targets, or the interaction between the targets and the targets, and edge information comprises the binding affinity, the binding loci and the interaction path.
